(19) United States
(12) Patent Application Publication
(10) Pub. No.: US 2009/0227521 A1
(43) Pub. Date: Sep. 10, 2009

(54) USE OF COMPOUNDS IN THE TREATMENT OF ISCHEMIA AND NEURODEGENERATION

(76) Inventors: Rickard Glas, Stockholm (SE); Hong Xu, Täby (SE)

Correspondence Address: FOX ROTHSCHILD LLP;PRINCETON PIKE CORPORATE CENTER, 2000 Market Street, Tenth Floor, Philadelphia, PA, 19103, US

(21) Appl. No.: 12/160,786

(22) PCT Filed: Jan. 15, 2007
(86) PCT No.: PCT/EP07/50364

§ 371 Date: Nov. 5, 2008

Related U.S. Application Data
(60) Provisional application No. 60/759,088, filed on Jan. 13, 2006.

(51) Int. Cl. A61K 38/06; A61P 25/28; C12Q 1/37

(52) U.S. Cl. 514/18; 435/24

(57) ABSTRACT

TPP II (tripeptidyl peptidase II) inhibitors are useful in the treatment of a neurodegenerative disease, for example Alzheimer's, Parkinson's or Huntingdon's disease or an ischemic condition, for example stroke and cardiac infarction. Suitable compounds comprise tripeptide compounds of general formula R^{N1}R^{N2}N-A¹-A²-A³-CO—R^{C1} wherein R^{N1}, R^{N2}, A¹, A², A³ and R^{C1} are as defined herein, and which include for example the tripeptide sequences GLA and GPG.

The present invention relates to the use of compounds in the treatment of ischemia and neurodegeneration.

Neurodegenerative diseases such as Alzheimer's, Parkinson's and Huntington's diseases progress slowly over a number of years, and finally require hospitalization and 24-hour attention, with huge medical costs. They may be caused by the deposition of protein aggregates that fail to be degraded, which ultimately leads to cell death in certain areas of the brain. There is a need for drugs that effectively treat these diseases.

There is a further need for effective methods of treating ischemia. In particular, during the acute phases of stroke and cardiac infarction there is massive cell death in the affected areas of the brain and in the heart muscle. Many researchers are trying to invent methods to protect cells in ischemic tissue, and to find effective drugs for this purpose.

The 26S proteasome recognizes substrates based on their conjugation to ubiquitin, and is responsible for the majority of cytosolic protein degradation. This process is important in the degradation of regulatory factors that control cell division, signal transduction, apoptosis and many other processes vital to multi-cellular organisms. Targeting of these regulatory factors for proteasomal degradation is determined by interactions with their ubiquitin conjugases. Pharmacological inhibition of proteasomal beta-sub-units arrests cell cycle progression with subsequent apoptosis, and also increases neo-synthesis of proteasomes. However, the level of proteasomal activity is not normally a rate-limiting step in substrate degradation and for control of cellular pathways. Several endogenous modulators of proteasomal biogenesis, e.g. PI31 and PA28, affect the specificity of proteasomal cleavage but they appear not to alter the rate of proteasomal protein degradation. Proteasomal 26S complexes are synthesized and assembled in the cytosol, and both 19S and 20S sub-complexes are imported into the nuclear compartment by Nuclear Localization Signal (NLS)-driven transport through the nuclear pores. Proteasomal complexes are distributed throughout the nucleus and cytosol, but a nuclear accumulation of proteasomes can occur in cells exposed to stress, which may alter proteasomal degradation of substrates. It is not known what causes such changes in sub-cellular distribution of proteasomal complexes.

Triggering of cellular stress response pathways require activation by Phosphoinositide-3-OH-kinase-related kinases (PIKKs), and among these are the ATM/ATR kinases essential for the stress response to DNA damage. Further, ATM kinase is also activated through ARK5 signaling in response to nutrient starvation. Impaired proteasomal activity and cellular stress are associated with the induction of complementary cytosolic peptidases, such as TPPII (tripeptidyl peptidase II) and iso-peptidases.

Several forms of stress interfere with the ubiquitin-proteasome pathway, including gamma-irradiation, ER-stress (ER=endoplasmic reticulum) and starvation. The level of inhibition is partial and may cause physiological effects, but the mechanisms behind many of these phenomena are unknown.

We now believe that ischemia and neurodegeneration are linked in terms of their reliance on downstream pathways of PIKKs, as further explained below, and can be treated by targeting a common mechanism. We have found a group of peptides and peptide-related compounds that are effective therapeutic agents. The present invention has arisen from our research into the role of TPP II (tripeptidyl-peptidase II). TPP II is built from a unique 138 kDa sub-unit expressed in multi-cellular organisms from Drosophila to Homo Sapiens. Data from Drosophila suggests that the TPP II complex consists of repeated sub-units forming two twisted strands with a native structure of about 6 MDa. TPP II is the only known cytosolic subtilisin-like serine peptidase. Bacterial subtilisins are thoroughly studied enzymes, with numerous reports on crystal structure and enzymatic function (Gupta, R., Beg, Q. K., and Lorenz, P., 2002, “Bacterial alkaline proteases: molecular approaches and industrial applications”, Appl Microbiol Biotechnol. 59:15-32).

Thus, from a first aspect the present invention provides a compound for use in the treatment of a neurodegenerative disease or an ischemic condition, wherein said compound is a TPP II inhibitor.

As used herein the term treatment covers the treatment of an established neurodegenerative disease or ischemic condition, as well as preventative therapy and the treatment of a pre-neurodegenerative or pre-ischemic condition.

From a further aspect the present invention provides a compound for use in the treatment of a neurodegenerative disease or an ischemic condition, wherein said compound is selected from the following formula (i) or is a pharmaceutically acceptable salt thereof:

R^{N1}R^{N2}N-A¹-A²-A³-CO—R^{C1} (i)
wherein A¹, A² and A³ are amino acid residues having the following definitions according to the standard one-letter abbreviations or names:
A¹ is G, A, V, L, I, P, 2-aminobutyric acid, norvaline or tert-butyl glycine,
A² is G, A, V, L, I, P, F, W, C, S, K, R, 2-aminobutyric acid, norvaline, norleucine, tert-butyl alanine, alpha-methyl leucine, 4,5-dehydro-leucine, allo-isoleucine, alpha-methyl valine, tert-butyl glycine, 2-allylglycine, ornithine or alpha, gamma-diaminobutyric acid,
A³ is G, A, V, L, I, P, F, W, D, E, Y, 2-aminobutyric acid, norvaline or tert-butyl glycine,
R^{N1} and R^{N2} are each attached to the N terminus of the peptide, are the same or different, and are each independently
   R^{N3},
   (linker1)-R^{N3},
   CO-(linker1)-R^{N3},
   CO—O-(linker1)-R^{N3},
   CO—N-((linker1)-R^{N3})R^{N4} or
   SO₂-(linker1)-R^{N3},
(linker1) may be absent, i.e. a single bond, or CH₂ CH₂CH₂, CH₂CH₂CH₂, CH₂CH₂CH₂CH₂ or CH═CH,
R^{N3} and R^{N4} are the same or different and are hydrogen or any of the following optionally substituted groups:
   saturated or unsaturated, branched or unbranched C₁₋₆ alkyl;
   saturated or unsaturated, branched or unbranched C₃₋₁₂ cycloalkyl;
   benzyl;
   phenyl;
   naphthyl;
   mono- or bicyclic C₁₋₁₀ heteroaryl; or
   non-aromatic C₁₋₁₀ heterocyclyl;
   wherein there may be zero, one or two (same or different) optional substituents on R^{N3} and/or R^{N4} which may be:
   hydroxy-;
   thio-:
   amino-;
   carboxylic acid;
   saturated or unsaturated, branched or unbranched C₁₋₆ alkyloxy;
   saturated or unsaturated, branched or unbranched C₃₋₁₂ cycloalkyl;
   N—, O—, or S— acetyl;
   carboxylic acid saturated or unsaturated, branched or unbranched C₁₋₆ alkyl ester;
   carboxylic acid saturated or unsaturated, branched or unbranched C₃₋₁₂ cycloalkyl ester
   phenyl;
   mono- or bicyclic C₁₋₁₀ heteroaryl;
   non-aromatic C₁₋₁₀ heterocyclyl; or
   halogen;
R^{C1} is attached to the C terminus of the tripeptide, and is:
   O—R^{C2},
   O-(linker2)-R^{C2},
   N((linker2)R^{C2})R^{C3}, or
   N(linker2)R^{C2}—NR^{C3}R^{C4},
(linker2) may be absent, i.e. a single bond, or C₁₋₆ alkyl or C₂₋₄ alkenyl, preferably a single bond or CH₂, CH₂CH₂, CH₂CH₂CH₂, CH₂CH₂CH₂CH₂ or CH═CH,
R^{C2}, R^{C3} and R^{C4} are the same or different, and are hydrogen or any of the following optionally substituted groups:
   saturated or unsaturated, branched or unbranched C₁₋₆ alkyl;
   saturated or unsaturated, branched or unbranched C₃₋₁₂ cycloalkyl;
   benzyl;
   phenyl;
   naphthyl;
   mono- or bicyclic C₁₋₁₀ heteroaryl; or
   non-aromatic C₁₋₁₀ heterocyclyl;
wherein there may be zero, one or two (same or different) optional substituents on each of R^{C2} and/or R^{C3} and/or R^{C4} which may be one or more of:
   hydroxy-;
   thio-:
   amino-;
   carboxylic acid;
   saturated or unsaturated, branched or unbranched C₁₋₆ alkyloxy;
   saturated or unsaturated, branched or unbranched C₃₋₁₂ cycloalkyl;
   N—, O—, or S— acetyl;
   carboxylic acid saturated or unsaturated, branched or unbranched C₁₋₆ alkyl ester;
   carboxylic acid saturated or unsaturated, branched or unbranched C₃₋₁₂ cycloalkyl ester
   phenyl;
   halogen,
   mono- or bicyclic C₁₋₁₀ heteroaryl; or
   non-aromatic C₁₋₁₀ heterocyclyl.

The N and CO indicated in the general formula for formula (i) are the nitrogen atom of amino acid residue A¹ and the carbonyl group of amino acid residue A³ respectively.

From a further aspect the invention provides a method of treatment of a neurodegenerative disease or an ischemic condition comprising administering to a patient in need thereof a therapeutically effective amount of a TPPII inhibitor or a compound selected from formula (i) or a pharmaceutically acceptable salt thereof.

Similarly, from a further aspect the present invention provides the use of a TPPII inhibitor or a compound selected from formula (i) or a pharmaceutically acceptable salt thereof in the manufacture of a medicament for the treatment of a neurodegenerative disease or an ischemic condition.

Without wishing to be bound by theory, the invention may be considered to recognize that TPP II inhibitors are useful in the treatment of a neurodegenerative disease or an ischemic condition.

From a further aspect the present invention provides a pharmaceutical composition comprising a compound of formula (i) or a pharmaceutically acceptable salt thereof and a pharmaceutically acceptable diluent or carrier.

From a further aspect the present invention provides a compound of formula (i) or a pharmaceutically acceptable salt thereof for use as a medicament.

From a further aspect the invention provides a method for identifying a compound suitable for the treatment of a neurodegenerative disease or an ischemic condition comprising contacting TPP II with a compound to be screened, and identifying whether the compound inhibits the activity of TPP II.

The present application claims priority from U.S. provisional patent application No. 60/759,088 filed 13 Jan. 2006 by inventors Rickard Glas and Hong Xu and entitled “Use of peptides and peptidomimetic compounds”, the contents of which are hereby incorporated in their entirety, insofar as that application relates to the treatment of ischemia and neurodegeneration.

The present invention recognizes an essential role for TPPII in down regulation of proteasomal substrate degradation in response to stress. As discussed in detail below, proteasomal complexes were translocated into the nucleus through a TPPII-dependent mechanism, which also required the activity of PIKK-family kinases. Blocking of PIKK-family kinases redistributed proteasomal complexes into the cytosol. We applied TPPII inhibitors, to increase degradation of otherwise degradation-resistant poly-Glutamine substrates, expressed in an in vitro cell line. Further, we showed that starvation-dependent accumulation of p53 and cell cycle arrest, was dependent on TPPII expression and activity. Our data support the use of inhibitors of TPPII in the treatment of neurodegenerative and ischemic diseases.

TPPII contributes to protein turnover of substrates, and was recently found to be the main peptidase to degrade cytosolic polypeptides longer than 15 amino acids (Reits, E., et. al. 2004. A major role for TPPII in trimming proteasomal degradation products for MHC class I antigen presentation. Immunity 20:495-506). Further TPPII can allow survival of lymphoma cells with inhibited proteasomal activity, which suggested a significant contribution to cellular protein turn-over. Our work shows that the involvement of TPPII in transduction of PIKK-family kinase activity also leads to an altered specificity of cytosolic proteolysis, since proteasomal substrate degradation was inhibited by TPPII. Thus in cells with normal proteasomal activity, TPPII is apparently working to restrain substrate degradation by the ubiquitin-proteasome pathway. It is presently not clear how the cellular level of proteasomal activity is controlled, and several reports suggest that modification of sub-units of the 19S proteasome could perform this role. Rad23, suggested to carry ubiquitinated proteins to the proteasome, interact with S2(Rpn1) through its ubiquitin-like (Ubl) domain (Elsasser S, et. al. 2002. Proteasome subunit Rpn1 binds ubiquitin-like protein domains. Nat Cell Biol. 4:725-30) (Zhang X, et. al. 2004. The targeting of the proteasomal regulatory subunit S2 by adenovirus E1A causes inhibition of proteasomal activity and increased p53 expression. J Biol. Chem. 279:25122-33). Furthermore, recent data suggest an ATP-driven dissociation of 19S and 20S complexes during each catalytic cycle showing that also 19S-20S re-association is regulated (Babbitt, S. E., et. al. 2005. ATP hydrolysis-dependent disassembly of the 26S proteasome is part of the catalytic cycle. Cell 121:553-65).

A reduced rate of proteasomal substrate degradation correlated with re-localization of proteasomal complexes, but our data do not exclude other mechanisms. Other potential mechanisms are direct modulation by PIKK-family members of proteasomal sub-units, e.g. Rpn10/S5a or other sub-units of the regulatory complex that are in direct contact with ubiquitinated substrates. The mechanism studied here may also have a role in DNA transcription and DNA repair, since nuclear proteasomal sub-complexes participate in these activities. A subset of TPPII is present in the nucleus, and it is possible that proteasomal complexes may be retained in this compartment, by a PIKK family kinase-TPPII-dependent mechanism. It can also not be excluded that down stream effectors regulate these events; such as mTOR or ARK5, a novel Akt-activated member of the AMPK family, that sense the cellular AMP/ATP levels.

The level of proteasomal activity is important when considering the therapy of neurodegenerative disease, but also p53 is important in causing neuronal apoptosis. Thereby, also other consequences of PIKK activation are of importance during this situation, and these are also important e.g. during ischemia, where p53 can be of importance for apoptosis in the affected tissue. Thus inhibitors of TPPII can be used to improve the therapy of diseases where increased proteasomal degradation is of benefit, such as in neurodegenerative diseases. In addition, the inhibition of p53 expression to transiently block transduction of stress signals leading to apoptosis, allows treatment of ischemia.

TPP II accepts a relatively broad range of substrates. All the compounds falling within formula (i) are peptides or peptide analogues. Compounds of formulae (i) are readily synthesizable by methods known in the art (see for example Ganellin et al., J. Med. Chem. 2000, 43, 664-674) or are readily commercially available (for example from Bachem A G). In a preferred aspect the compound may be selected from formulae (i). Such tripeptides and derivatives are particularly effective therapeutic agents.

According to the invention the compound for use in the treatment of a neurodegenerative disease or an ischemic condition may be a compound which is known to be a TPP II inhibitor in vivo.

For example, the compound may be selected from compounds identified in Winter et al., Journal of Molecular Graphics and Modelling 2005, 23, 409-418 as TPP II inhibitors. The compounds may be selected from the following formula (ii) because these compounds are particularly suited to the TPP II pharmacophore.

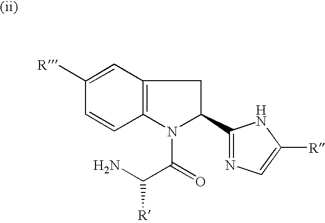

wherein R′ is H, CH₃, CH₂CH₃, CH₂CH₂CH₃ or CH(CH₃)₂,
R″ is H, CH₂CH₃, CH₂CH₂CH₃, CH(CH₃)₂, CH₂CH₂CH₂CH₃, CH₂CH(CH₃)₂, CH(CH₃)CH₂CH₃ or C(CH₃)₃, and
R′″ is H, CH₃, OCH₃, F, Cl or Br;

Compounds of formula (ii) are synthesizable by known methods (see for example Winter et al., Journal of Molecular Graphics and Modelling 2005, 23, 409-418 and Breslin et al., Bioorg. Med. Chem. Lett. 2003, 13, 4467-4471).

Also by way of example, the compound may be selected from compounds identified in U.S. Pat. No. 6,335,360 of Schwartz et al. as TPP II inhibitors. Such compounds include those of the following formula (iii).

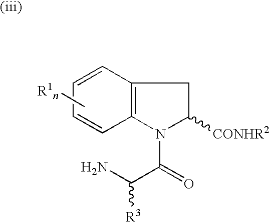

wherein:
each R¹ may be the same or different, and is selected from the group consisting of halogen, OH; C₁-C₆ alkyl optionally substituted by one or more radicals selected from the group consisting of halogen and OH; (C₁-C₆) alkenyl optionally substituted by one or more radicals selected from the group consisting of halogen and OH; (C1-C₆) alkynyl, optionally substituted by one or more radicals selected from the group consisting of halogen and OH, X(C₁-C₆)alkyl, wherein X is S, 0 or OCO, and the alkyl is optionally substituted by one or more radicals selected from the group consisting of halogen and OH; SO₂ (C₁-C₆)alkyl, optionally substituted by at least one halogen, YSO₃H, YSO₂ (C₁-C₆)alkyl, wherein Y is O or NH and the alkyl is optionally substituted by at least one halogen, a diradical —X1-(C₁-C₂)alkylene-X1-wherein X₁ is O or S; and a benzene ring fused to the indoline ring;
n is from 0 to 4;
R² is CH₂R⁴, wherein R⁴ is C₁-C₆ alkyl substituted by one or more radicals selected from the group consisting of halogen and OH; (CH₂)ₚZ(CH₂)_{q}CH₃, wherein Z is O or S, p is from 0 to 5 and q is from 0 to 5, provided that p+q is from 0 to 5; (C₂-C₆) unsaturated alkyl; or (C₃-C₆) cycloalkyl;
or R² is (C₁-C₆)alkyl or O(C₁-C₆)alkyl, each optionally substituted by at least one halogen;
R³ is H; (C₁-C₆)alkyl optionally substituted by at least one halogen; (CH₂)ₚZR⁵ wherein p is from 1 to 3, Z is O or S and R⁵ is H or (C₁-C₃)alkyl; benzyl.

Compounds of formula (iii) are readily synthesizable by known methods (see for example U.S. Pat. No. 6,335,360 of Schwartz et al.).

Nevertheless, it is preferred that the compound be selected from formulae (i) and (ii), more preferably formula (i).

It is also possible for the compound to be a compound of formula (i) wherein R^{N1}, R^{N2} and R^{C1} are as defined above or in any of the preferences below and wherein:
A¹ is G, A, V, L, I, P, S, T, C, N, Q, 2-aminobutyric acid, norvaline, norleucine, tert-butyl alanine, alpha-methyl leucine, 4,5-dehydro-leucine, allo-isoleucine, alpha-methyl valine, tert-butyl glycine or 2-allylglycine,
A² is G, A, V, L, I, P, S, T, C, N, Q, F, Y, W, K, R, histidine, 2-aminobutyric acid, norvaline, norleucine, tert-butyl alanine, alpha-methyl leucine, 4,5-dehydro-leucine, allo-isoleucine, alpha-methyl valine, tert-butyl glycine, 2-allylglycine, ornithine, alpha, gamma-diaminobutyric acid or 4,5-dehydro-lysine, and
A³ is G, A, V, L, I, P, S, T, C, N, Q, D, E, F, Y, W, 2-aminobutyric acid, norvaline, norleucine, tert-butyl alanine, alpha-methyl leucine, 4,5-dehydro-leucine, allo-isoleucine, alpha-methyl valine, tert-butyl glycine or 2-allylglycine.

### Preferred Compounds of Formula (i)

Various groups and specific examples of compounds of formula (i) are preferred.

In general, amino acids of natural (L) configuration are preferred, particularly at the A² position.

In general, it is preferred that R^{N1} is hydrogen, and that
R^{N2} is:
   R^{N3},
   (linker1)-R^{N3},
   CO-(linker1)-R^{N3}, or
   CO—O-(linker1)-R^{N3},
wherein
(linker1) may be absent, i.e. a single bond, or CH₂, CH₂CH₂, CH₂CH₂CH₂, CH₂CH₂CH₂CH₂ or CH═CH, and
R^{N3} is hydrogen or any of the following unsubstituted groups:
   saturated or unsaturated, branched or unbranched C₁₋₄ alkyl;
   benzyl;
   phenyl; or
   monocyclic heteroaryl.
In general, it is preferred that R^{C1} is:
   O—R^{C2},
   O-(linker2)-R^{C2}, or
   NH-(linker2)R^{C2}
wherein
(linker2) may be absent, i.e. a single bond, C₁₋₆ alkyl or C₂₋₄ alkenyl, preferably a single bond or CH₂, CH₂CH₂, CH₂CH₂CH₂, CH₂CH₂CH₂CH₂ or CH═CH,
R^{C2} is hydrogen or any of the following unsubstituted groups:
   saturated or unsaturated, branched or unbranched C₁₋₅ alkyl;
   benzyl;
   phenyl; or
   monocyclic C₁₋₁₀ heteroaryl.

In general, with regard to the substituents at the N-terminus, it is further preferred that:

R^{N1} is hydrogen, and
R^{N2} is hydrogen, C(═O)—O-(linker1)-R^{N3} or C(═O)-(linker1)-R^{N3},
(linker1) is CH₂ or CH═CH, and
R^{N3} is phenyl or 2-furyl.

It is further preferred that

R^{N1} is hydrogen,
R^{N2} is hydrogen, C(═O)—OCH₂Ph or C(═O)—CH═CH-(2-furyl).

Another preferred grouping for the substituents on the N-terminus is such that:

R^{N1} is hydrogen, and
R^{N2} is a is benzyloxycarbonyl, benzyl, benzoyl, tert-butyloxycarbonyl, 9-fluorenylmethoxycarbonyl or FA, more preferably benzyloxycarbonyl or FA.

In general, with regard to the substituents at the C-terminus, it is preferred that:

R^{C1} is OH, O—C₁₋₆ alkyl, O—C₁₋₆ alkyl-phenyl, NH—C₁₋₆ alkyl, or NH—C₁₋₆ alkyl-phenyl, more preferably OH.

Several preferred groups are as follows.

Group (i)(a):
A¹ is G, A, V, L, I, P, 2-aminobutyric acid, norvaline or tert-butyl glycine,
A² is G, A, V, L, I, P, F, W, C, S, K, R, 2-aminobutyric acid, norvaline, norleucine, tert-butyl alanine, alpha-methyl leucine, 4,5-dehydro-leucine, allo-isoleucine, alpha-methyl valine, tert-butyl glycine, 2-allylglycine, ornithine or alpha, gamma-diaminobutyric acid,
A³ is G, A, V, L, I, P, F, W, D, E, Y, 2-aminobutyric acid, norvaline or tert-butyl glycine,

### R^{N1} is H,

R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.
Group (i)(b):
A¹ is G, A or 2-aminobutyric acid,
A² is L, I, norleucine, V, norvaline, tert-butyl alanine, 4,5-dehydro-leucine, allo-isoleucine, 2-allylglycine, P, 2-aminobutyric acid, alpha-methyl leucine, alpha-methyl valine or tert-butyl glycine,
A³ is G, A, V, P, 2-aminobutyric acid or norvaline,

### R^{N1} is H,

R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.
Group (i)(c):
A¹ is G, A or 2-aminobutyric acid,
A² is L, I, norleucine, V, norvaline, tert-butyl alanine, 4,5-dehydro-leucine, allo-isoleucine or 2-allylglycine,
A³ is G, A, V, P, 2-aminobutyric acid or norvaline,

### R^{N1} is H,

R^{N2} is hydrogen, C(═O)—O-saturated or unsaturated, branched or unbranched, C₁₋₄ alkyl optionally substituted with phenyl or 2-fury, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—CO₁₋₆ alkyl, or NH—C₁₋₆ alkyl-phenyl.
Group (i)(d):

### A¹ is G or A,

A² is L, I, or norleucine,

### A³ is G or A,

### R^{N1} is H,

R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.

A first set of specific preferred compounds are those in which:

### A¹ is G,

### A² is L,

A³ is G, A, V, L, I, P, F, W, D, E, Y, 2-aminobutyric acid, norvaline or tert-butyl glycine, more preferably G, A, V, P, 2-aminobutyric acid or norvaline, more preferably G or A,
R^{N1} is hydrogen,
R^{N2} is benzyloxycarbonyl, and

### R^{C1} is OH.

A second set of specific preferred compounds are those in which:

### A¹ is G,

A² is G, A, V, L, I, P, F, W, C, S, 2-aminobutyric acid, norvaline, norleucine, tert-butyl alanine, alpha-methyl leucine, 4,5-dehydro-leucine, allo-isoleucine, alpha-methyl valine, tert-butyl glycine or 2-allylglycine, more preferably L, I, norleucine, V, norvaline, tert-butyl alanine, 4,5-dehydro-leucine, allo-isoleucine, 2-allylglycine, P, 2-aminobutyric acid, alpha-methyl leucine, alpha-methyl valine or tert-butyl glycine, more preferably L, 3, norleucine, V, norvaline, tert-butyl alanine, 4,5-dehydro-leucine, allo-isoleucine or 2-allylglycine, more preferably L, I, or norleucine,

### A³ is A,

R^{N1} is hydrogen,
R^{N2} is benzyloxycarbonyl, and

### R^{C1} is OH.

A third set of specific preferred compounds are those in which:

A¹ is G, A, V, L, I, P, 2-aminobutyric acid, norvaline or tert-butyl glycine, more preferably G, A or 2-aminobutyric acid, more preferably G or A,

### A² is L,

### A³ is A,

R^{N1} is hydrogen,
R^{N2} is benzyloxycarbonyl, and

### R^{C1} is OH.

Preferably the sequence A¹-A²-A³ is GLA, GLF, GVA, GIA, GPA or ALA, most preferably GLA, and:

R^{N1} is hydrogen,
R^{N2} is benzyloxycarbonyl, and

### R^{C1} is OH.

Where alkyl groups are described as saturated or unsaturated, this encompasses alkyl, alkenyl and alkynyl hydrocarbon moieties.

C₁₋₆ alkyl is preferably C₁₋₄ alkyl, more preferably methyl, ethyl, n-propyl, isopropyl, or butyl (branched or unbranched), most preferably methyl.

C₃₋₁₂ cycloalkyl is preferably C₅₋₁₀ cycloalkyl, more preferably C₅₋₇ cycloalkyl.

“aryl” is an aromatic group, preferably phenyl or naphthyl,

“hetero” as part of a word means containing one or more heteroatom(s) preferably selected from N, O and S.

“heteroaryl” is preferably pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, thiophenetyl, pyranyl, carbazolyl, acridinyl, quinolinyl, benzimidazolyl, benzthiazolyl, purinyl, cinnolinyl or pteridinyl.

“non-aromatic heterocyclyl” is preferably pyrrolidinyl, piperidyl, piperazinyl, morpholinyl, tetrahydrofuranyl or monosaccharide.

“halogen” is preferably Cl or F, more preferably Cl.

### Further Preferred Compounds of Formula (i)

In general, A¹ may preferably be selected from G, A or 2-aminobutyric acid; more preferably G or A.

In general, A² may preferably be selected from L, I, norleucine, V, norvaline, tert-butyl alanine, 4,5-dehydro-leucine, allo-isoleucine, 2-allylglycine, P, K, 2-aminobutyric acid, alpha-methyl leucine, alpha-methyl valine or tert-butyl glycine; more preferably L, I, norleucine, V, norvaline, tert-butyl alanine, 4,5-dehydro-leucine, allo-isoleucine, 2-allylglycine, P or K; more preferably L, I, norleucine, P or K; more preferably L or P.

In general, A³ may preferably be selected from G, A, V, P, 2-aminobutyric acid or norvaline; more preferably G or A.

In general, it is preferred that R^{N1} is hydrogen.

In general, R^{N2} is preferably:
R^{N3},
(linker1)-R^{N3},
CO-(linker1)-R^{N3}, or
CO—O-(linker1)-R^{N3},
wherein
(linker1) may be absent, i.e. a single bond, or CH₂, CH₂CH₂, CH₂CH₂CH₂, CH₂CH₂CH₂CH₂ or CH═CH, and
R^{N3} is hydrogen or any of the following unsubstituted groups:
   saturated or unsaturated, branched or unbranched C₁₋₄ alkyl,
   benzyl;
   phenyl; or
   monocyclic heteroaryl.

In general, R^{N2} is more preferably hydrogen, benzyloxycarbonyl, benzyl, benzoyl, tert-butyloxycarbonyl, 9-fluorenylmethoxycarbonyl or FA, more preferably hydrogen, benzyloxycarbonyl or FA.

In general, it is preferred that R^{C1} is:
O—R^{C2},
O-(linker2)-R^{C2}, or
NH-(linker2)R^{C2}
wherein
(linker2) may be absent, i.e. a single bond, C₁₋₆ alkyl or C₂₋₄ alkenyl, preferably a single bond or CH₂, CH₂CH₂, CH₂CH₂CH₂, CH₂CH₂CH₂CH₂ or CH═CH,
R^{C2} is hydrogen or any of the following unsubstituted groups:
   saturated or unsaturated, branched or unbranched C₁₋₅ alkyl;
   benzyl;
   phenyl; or
   monocyclic C₁₋₁₀ heteroaryl.

In general, R^{C1} is more preferably OH, O—C₁₋₆ alkyl, O—C₁₋₆ alkyl-phenyl, NH₂, NH—C₁₋₆ alkyl, or NH—C₁₋₆ alkyl-phenyl, more preferably OH, O—C₁₋₆ alkyl, NH₂, or NH—C₁₋₆ alkyl, more preferably OH or NH₂.

Compounds of particular interest include those wherein A² is P.

Compounds of particular interest include those wherein R^{C1} is NH₂.

In general the following amino acids are less preferred for A³: F, W, D, E and Y. Similarly, in general A³ may be selected not to be P and/or E due to compounds containing these exhibiting lower activity.

### Preferred Compounds of Formula (ii)

Compounds of formula (ii) are preferably such that:

### R′ is CH₂CH₃ or CH₂CH₂CH₃,

### R″ is CH₂CH₂CH₃ or CH(CH₃)₂, and

### R′″ is H or Cl.

Preferred Compounds of Formula (iii)

Various preferred groups and specific examples of compounds of formula (iii) are as defined in any of the claims, taken separately, of U.S. Pat. No. 6,335,360 B1 of Schwartz et al.

One example of a therapeutic compound of formula (i) is Z-GLA-OH, i.e. tripeptide GLA which is derivatized at the N-terminus with a Z group and which is not derivatized at the C-terminus. Z denotes benzyloxycarbonyl. This is a compound of formula (i) wherein R^{N1} is H, R^{N2} is Z, A¹ is G, A² is L, A³ is A and R^{C1} is OH. This compound is available commercially from Bachem AG and has been found to inhibit the bacterial homologue of the eukaryotic TPP II, Subtilisin. Z-GLA-OH is of low cost and works well experimentally.

Whilst preferred compounds include those containing GLA, such as Z-GLA-OH, Bn-GLA-OH, FA-GLA-OH and H-GLA-OH, for example Z-GLA-OH; according to the present invention any disclosures of any compounds or groups of compounds herein may optionally be subject to the proviso that the sequence A¹A²A³ is not GLA, or the proviso that the compound is not selected from the group consisting of Z-GLA-OH, Bn-GLA-OH, FA-GLA-OH or H-GLA-OH, or the proviso that the compound is not Z-GLA-OH.

In the treatment of a neurodegenerative disease or an ischemic condition Z-GLA-OH or other compounds described herein may be administered.

Other preferred compounds include those wherein A¹A²A³ is GPG, such as GPG-NH₂ or Z-GPG-NH₂.

The skilled person will be aware that the compounds described herein may be administered in any suitable manner. For example, the administration may be parenteral, such as intravenous or subcutaneous, oral, transdermal, intranasal, by inhalation, or rectal. In one preferred embodiment the compounds are administered by injection.

Examples of pharmaceutically acceptable addition salts for use in the pharmaceutical compositions of the present invention include those derived from mineral acids, such as hydrochloride hydrobromic, phosphoric, metaphosphoric, nitric and sulphuric acids, and organic acids, such as tartaric, acetic, citric, malic, lactic, fumaric, benzoic glycolic, gluconic, succinic, and arylsulphonic acids. The pharmaceutically acceptable excipients described herein, for example, vehicles, adjuvants, carriers or diluents, are well-known to those who are skilled in the art and are readily available to the public. The pharmaceutically acceptable carrier may be one that is chemically inert to the active compounds and that has no detrimental side effects or toxicity under the conditions of use. Pharmaceutical formulations are found e.g. in Remington: The Science and Practice of Pharmacy, 19th ed., Mack Printing Company, Easton, Pa. (1995).

The composition may be prepared for any route of administration, e.g. oral, intravenous, cutaneous or subcutaneous, nasal, intramuscular, or intraperitoneal. The precise nature of the carrier or other material will depend on the route of administration. For a parenteral administration, a parenterally acceptable aqueous solution is employed, which is pyrogen free and has requisite pH, isotonicity and stability. Those skilled in the art are well able to prepare suitable solutions and numerous methods are described in the literature. A brief review of methods of drug delivery is also found in e.g. Langer, Science 249:1527-1533 (1990).

The dose administered to a mammal, particularly a human, in the context of the present invention should be sufficient to effect a therapeutic response in the mammal over a reasonable time frame. One skilled in the art will recognize that dosage will depend upon a variety of factors including the age, condition and body weight of the patient, as well as the stage/severity of the disease. The dose will also be determined by the route (administration form) timing and frequency of administration. In the case of oral administration the dosage can vary for example from about 0.01 mg to about 10 g, preferably from about 0.01 mg to about 1000 mg, more preferably from about 10 mg to about 1000 mg per day of a compound or the corresponding amount of a pharmaceutically acceptable salt thereof.

Treatment may be applied in a single dose, or periodically as a course of treatment.

It is clear to the skilled person how to screen compounds for their inhibition of the activity of TPP II. TPP II protein may be purified in a first step, and a TPP II-preferred fluorogenic substrate may be used in a second step. This results in an effective method to measure TPP II activity.

It is not necessary to achieve a particularly high level of purification, and conventional simple techniques can be used to obtain TPP II of sufficient quality to use in a screening method. In one non-limiting example of purification of TPP II, 100×10⁶ cells (such as EL-4 cells) were sedimented and lysed by vortexing in glass beads and homogenisation buffer (50 mM Tris Base pH 7.5, 250 mM Sucrose, 5 mM MgCl₂, 1 mM DTT). Cellular lysates were subjected to differential centrifugation; first the cellular homogenate was centrifuged at 14,000 rpm for 15 min, and then the supernatant was transferred to ultra-centrifugation tubes. Next the sample was ultra-centrifugated at 100,000×g for 1 hour, and the supernatant (denoted as cytosol in most biochemical literature) was subjected to 100,000×g centrifugation for 5 hours, which sedimented high molecular weight cytosolic proteins/protein complexes. The resulting pellet dissolved in 50 mM Tris Base pH 7.5, 30% Glycerol, 5 mM MgCl₂, and 1 mM DTT, and 1 ug of high molecular weight protein was used as enzyme in peptidase assays.

It is possible to test the activity of TPP II using for example the substrate AAF-AMC (Sigma, St. Louis, Mo.). This may for example be used at 100 uM concentration in 100 ul of test buffer composed of 50 mM Tri Base pH 7.5, 5 mM MgCl₂ and 1 mM DTT. It is possible to stop reactions using dilution with 900 ul 1% SDS solution. Cleavage activity may be measured for example by emission at 460 nm in a LS50B Luminescence Spectrometer (Perkin Elmer, Boston, Mass.).

The compounds of use in the present invention may be defined as those which result in partial or preferably complete treatment of ischemia or neurodegeneration in vivo.

The compounds used in the present invention are sufficiently serum-stable, i.e. in vivo they retain their identity long enough to exert the desired therapeutic effect.

Signal transduction of several forms of stress depends on enzymes of the PI3K-like kinase family (PIKKs). These include nuclear enzymes ATM, ATR and DNA-PKcs and also mTOR in the cytosol. Our data have supported that PIKKs contribute to the stabilization of Tripeptidyl-peptidase II (TPPII), a high molecular weight peptidase in the cytosol. Further, TPPII appears necessary for several of the downstream pathways of PIKKs, such as p53 stabilization and resistance to gamma-irradiation in vivo. Several forms of stress inhibit the activity of the ubiquitin-proteasome pathway (UPP), and our results show that Tripeptidyl-peptidase II (TPPII, a large cytosolic peptidase) caused inhibition of the UPP during cellular stress. Reduced UPP activity coincided with translocation of proteasomal complexes into the nucleus, and nuclear translocation of proteasomes was dependent on the activity of PI3K-like kinases (PIKKs). PIKK activity was required for protection of TPPII from proteasomal degradation, and inhibited expression or activity of TPPII prevented stress-induced nuclear localization of proteasomes. Inhibitors of TPPII accelerated the proteasomal degradation of otherwise degradation-resistant poly-Glutamine substrates. Our data suggest that TPPII mediated suppression of proteasomal substrate degradation and relocalization of proteasomal complexes is a consequence of PIKK activation in response to cellular stress. This leads to the use of TPPII inhibitors in the treatment of neurodegenerative diseases.

In addition, our results show that TPPII is strongly up-regulated in response to starvation, an event that was also dependent on the activity of PIKKs. As found in response to gamma-irradiation, TPPII was also important for p53 accumulation in response to starvation. p53 is a strong determinant for pathology in certain ischemic diseases, whereby inhibition of TPPII may be used to treat ischemic diseases.

The present invention is described in more detail in the non-limiting Examples below with reference to the accompanying drawings which are now summarised.

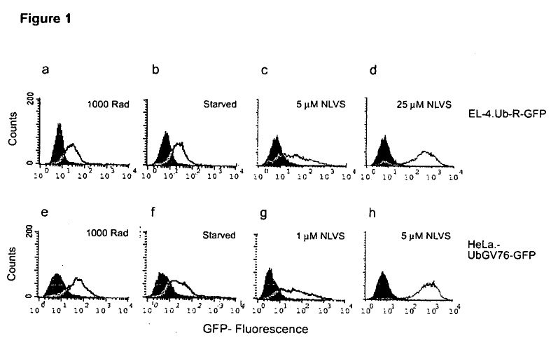

FIG. 1. Suppression of proteasomal substrate degradation during cellular stress. (a-h) GFP-fluorescence as quantified by flow cytometry of EL-4.Ub-R-GFP (a-d) and HeLa.UbGV76-GFP cells (e-h) cells exposed to gamma-irradiation, starvation or treatment with proteasomal inhibitor, as indicated. Gamma-irradiated cells were exposed to 1000 Rad and incubated in vitro for 3-4 days. Starved cells were grown in dense standard in vitro cultures without replenishing medium for 5-7 days. Dead cells were excluded by gating with Propidium Iodide (PI).

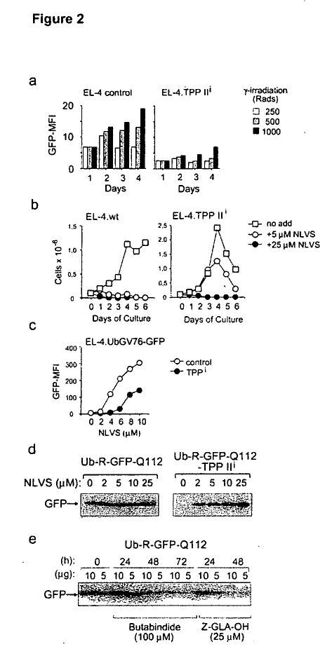

FIG. 2. Stress-induced suppression of the ubiquitin-proteasome pathway depends on TPPII. (a) Mean fluorescence intensity of EL-4.UbGV76-GFP (left) and EL-4.UbGV76-GFP/TPPIIⁱ cells 1-4 days after exposure to gamma-irradiation. (b) Cellular growth in vitro of EL-4.wt and EL-4.TPPIIⁱ cells in the presence of 0, 5 or 25 micro-M of NLVS. (c) Mean fluorescence intensity (MFI) as quantified by flow cytometry of EL-4.UbGV76-GFP (empty circles) and EL-4.UbGV76-GFP/TPPIIⁱ cells (filled circles), treated with 0, 2, 4, 6, 8 or 10 micro-M NLVS overnight. (d) Ub-R-GFP-Q112 expression in stably transfected EL-4 versus EL-4.TPPIIⁱ cells, either left untreated or treated with the indicated concentration with NLVS. (e) Ub-R-GFP-Q112 expression in stably transfected EL-4 cells treated with either Butabindide or the TPPII inhibitor Z-GLA-OH, for up to 72 hours.

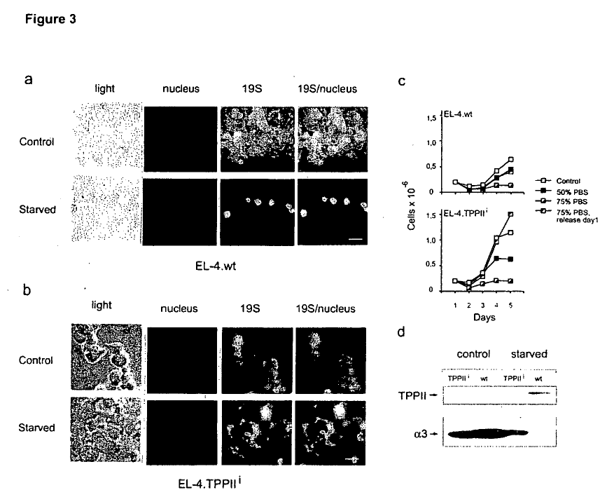

FIG. 3. Nuclear localization of proteasomes during cellular stress depends on TPPII. (a, b) 19S proteasome location in EL-4.wt (a) versus EL-4.TPPIIⁱ (b), comparing untreated (top panels) versus starved (bottom panels) cells, as detected by staining with anti-Rpt6 (19S base sub-unit). Scale bar=5 micro-m. (c) In vitro proliferation of EL-4.wt (top) versus EL-4.TPPIIⁱ cells (bottom), exposed to starvation for the indicated time periods. (d) Western blotting with anti-TPPII and anti-proteasome alpha-3 of cytosolic fractions from EL-4.wt and EL-4.TPPIIⁱ cells, exposed to starvation or left untreated.

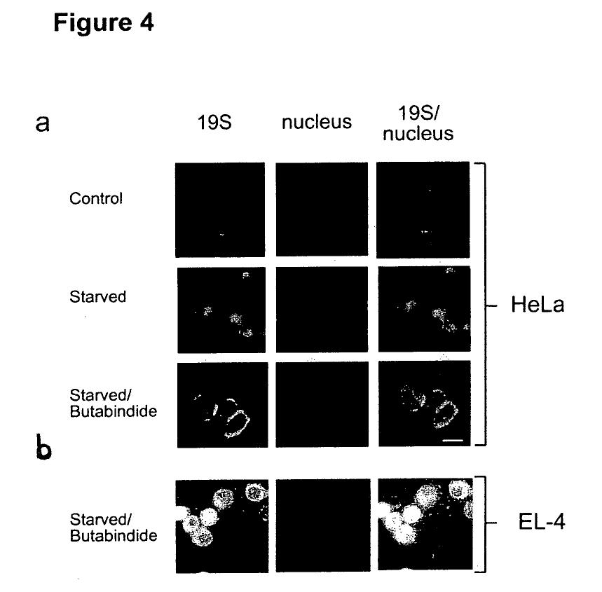

FIG. 4. TPPII inhibition prevents nuclear localization of proteasomes during stress. (a) 19S proteasome location in HeLa cells left untreated (top), exposed to starvation (middle) or exposed to starvation and 100 micro-M Butabindide (bottom). Scale bar=5 micro-m. (b) 19S proteasome location in starved EL-4 cells, as detected by staining for Rpt6, in the presence of 100 micro-M Butabindide.

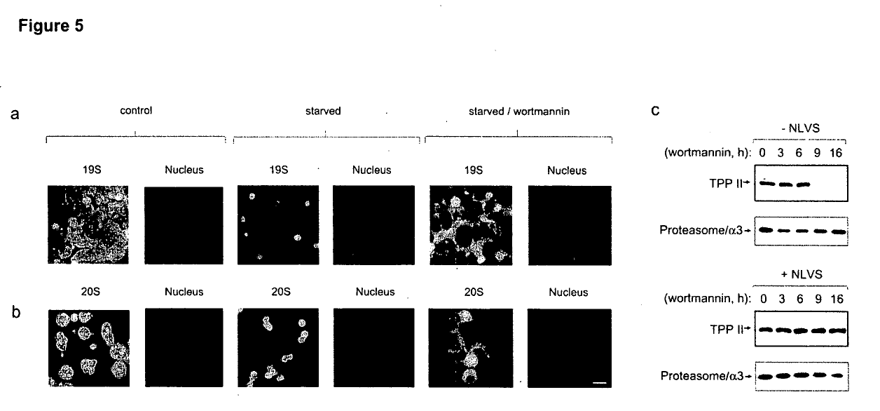

FIG. 5. PIKK-family kinase activity controls TPPII expression and nuclear localization of proteasomes. (a, b) Immunohistochemical analysis of EL-4 cells exposed to starvation in the presence or absence of 1 micro-M wortmannin (inhibitor of PIKK-family kinases), and stained for anti-Rpt6 (19S proteasome, a) or anti-alpha-3 (20S proteasome, b). Scale bar=5 micro-m. (c) Western blotting for anti-TPPII and anti-protetasome alpha-3 in starved EL-4 cells treated with 1 micro-M wortmannin for 0, 3, 6, 9 and 16 hours.

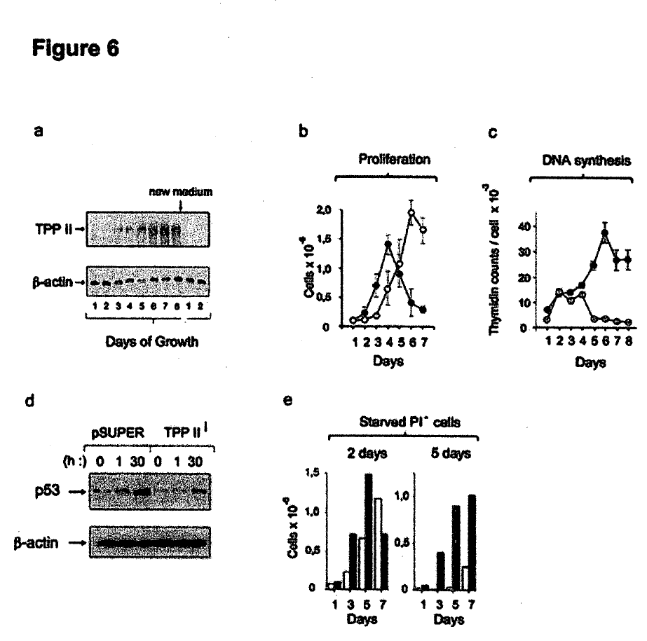

FIG. 6. Starvation-induced cell cycle arrest requires TPP II expression.

(a) Western blotting with anti-TPP II of cellular lysates derived from EL-4.wt control cells seeded at 10⁵ cells/ml at day 1, and cultured for 8 days in the absence of cell culture medium exchange. The arrow indicates the addition of fresh cell culture medium. (b, c) Live cells (b) and DNA synthesis (c) during in vitro culture of EL-4.wt and EL-4.TPP IIⁱ cells. (d) p53 expression in EL-4.wt control versus EL-4.TPP IIⁱ cells exposed to starvation in 60% PBS, for 0, 1 or 30 hours, (e) Live EL4.wt (empty bars) and EL-4.TPP IIⁱ cells (filled bars), growing in normal tissue culture medium; after culture in starvation medium for 2 or 5 days.

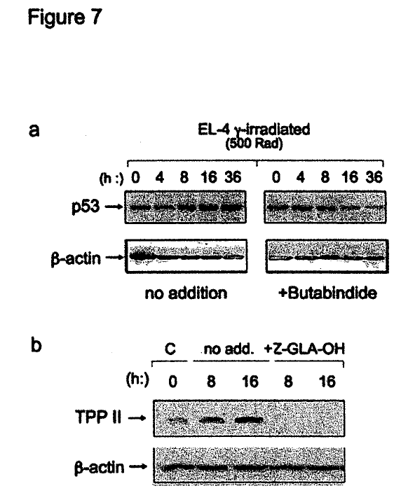

FIG. 7. TPPII inhibitors prevent expression of TPPII protein and stabilization of p53. (a) Western blot analysis of p53 in gamma-irradiated (500 Rad) EL-4.wt cells treated with 100 micro-M of butabindide from 2 hours before experiment onset, or left untreated. (b) Western blot analysis of TPP II in gamma-irradiated (500 Rad) EL-4.wt control cells, treated with 25 micro-M Z-GLA-OH or left untreated.

### EXAMPLES

The materials and methods used were as follows.

Cells and Culture Conditions. EL-4 is a Benzpyrene-induced lymphoma cell line derived from the C57BI/6 mouse strain. EL-4.wt and EL-4.TPPIIⁱ are EL-4 cells transfected with the pSUPER vector (Brummelkamp, T R, Bernards, R, Agami, R. A system for stable expression of short interfering RNAs in mammalian cells. Science 2002; 296:550-3), empty versus containing the siRNA directed against TPPII. HeLa cells are human cervical carcinoma cells. For induction of stress, cells were starved by growth in 50%-75% Phosphate Buffered Saline (PBS) or gamma-irradiated 250-2000 Rad's; and incubated at 37° C. and 5.3% CO₂. Our flow cytometric data represent live cells as determined by flow cytometric gating with Propidium Iodide (PI). For generation of stable transfectants, 5×10⁶ cells were washed in PBS, then resuspended into 500 micro-I of PBS in a Bio-Rad gene-pulser and pulsed with 10 micro-g DNA and 250 V at 960 micro-F; and selected by resistance to G418. UbGV76-GFP is a ubiquitin-fusion construct that creates a rapidly degraded GFP molecule for monitoring 26S proteasome activity in live cells through flow cytometry (Dantuma, N. P, Lindsten, K., Glas, R., Jellne, M., and Masucci, M. G. 2000. Short-lived green fluorescent proteins for quantifying ubiquitin/proteasome-dependent proteolysis in living cells. Nat. Biotechnol. 18:538-43). Ub-R-GFP is a similar vector that also encodes a highly unstable GFP molecule degraded through the ubiquitin-proteasome pathway. Ub-R-GFP-Q112 is the same substrate but contains an extended C-terminal with 112 glutamines (Verhoef, L. G., Lindsten, K., Masucci, M. G. and Dantuma, N. P. 2002. Aggregate formation inhibits proteasomal degradation of polyglutamine proteins. Hum Mol. Genet. 11:2689-700).

Enzyme Inhibitors. NLVS is an inhibitor of the proteasome that preferentially targets the chymotryptic peptidase activity, and efficiently inhibits proteasomal degradation in live cells. Butabindide is described in the literature (Rose, C, Vargas, F, Facchinetti, P, Bourgeat, P, Bambal, R B, Bishop, P B, et. al. Characterization and inhibition of a cholecystokinin-inactivating serine peptidase. Nature 1996; 380:403-9). Z-Gly-Leu-Ala-OH (Z-GLA-OH) is an inhibitor of Subtilisin (Bachem, Weil am Rhein, Germany), a bacterial enzyme with an active site that is homologous to that of TPPII. Wortmannin is an inhibitor of PIKK (PI3-kinase-related)-family kinases (Sigma, St. Louis, Mo.). All inhibitors were dissolved in DMSO and stored at −20° C. until use.

Protein Purification, Peptidase Assays and Analysis of DNA Fragmentation. 100×10⁶ cells were sedimented and lysed by vortexing in glass beads and homogenisation buffer (50 mM Tris Base pH 7.5, 250 mM Sucrose, 5 mM MgCl2, 1 mM DTT). Cellular lysates were submitted to differential centrifugation where a supernatant from a 1 hour centrifugation at 100,000×g (cytosol) was submitted to 100,000×g centrifugation for 3-5 hours, which sedimented high molecular weight cytosolic proteins/protein complexes. The resulting pellet dissolved in 50 mM Tris Base pH 7.5, 30% Glycerol, 5 mM MgCl2, and 1 mM DTT, and 1 micro-g of high molecular weight protein was used as enzyme in peptidase assays or in Western blotting for TPP II expression. To test the activity of TPP II we used the substrate AAF-AMC (Sigma, St. Louis, Mo.), at 100 micro-M concentration in 100 micro-I of test buffer composed of 50 mM Tri Base pH 7.5, 5 mM MgCl2 and 1 mM DTT. Cleavage activity was measured by emission at 460 nm in a LS50B Luminescence Spectrometer (Perkin Elmer, Boston, Mass.). For analysis of DNA fragmentation cells were seeded in 12-well plates at 10⁶ cells/ml and exposed to 25 micro-M etoposide, a DNA topoisomerase II inhibitor commonly used as an apoptosis-inducing agent, to starvation (50% PBS). Cells were seeded at 10⁶ cells/ml in 12-well plates and incubated for the indicated times, usually 18-24 hours. DNA from EL-4 control and adapted cells was purified by standard chloroform extraction, and 2.5 micro-g of DNA was loaded on 1.8% agarose gel for detection of DNA from apoptotic cells.

Antibodies and Antisera. The following molecules were detected by the antibodies specified: GFP by rabbit anti-GFP serum (Molecular Probes Europe, Breda, The Netherlands); 19S proteasomal complexes by anti-Rpt6 (19S base ATPase subunit), 20S proteasomal complexes by (Affinity, Exeter, UK); For detection of TPPII we used chicken anti-TPPII serum (Immunsystem, Uppsala, Sweden). In experiments where whole cell lysates were used for western blotting of TPPII, i.e. fractions not enriched for TPPII, TPPII fell below the limit of detection in cells not exposed to stress. Western blotting was performed by standard techniques. Protein concentration was measured by BCA Protein Assay Reagent (Pierce Chemical Co.). 5 micro-g of protein was loaded per lane for separation by SDS/PAGE unless stated otherwise.

Immunohistochemistry Cells were attached to glass cover slips through cytospin and fixed in acetone-methanol (1:1) for 1 hour; then the slides were rehydrated in BSS buffer for 1 hour. The first antibody was added and remained for 1 hour until a brief wash in BSS, after which a secondary conjugate (anti-rabbit-FITC) was added and incubated for 1 hour. Then the slides were washed and stained with Hoescht 333258 for 30 min. Finally, the slides were mounted with DABCO mounting buffer and kept at 4° C. until analysis.

Flow Cytometry. Fluorescence was quantified by a FACScalibur. Flow cytometric cell sorting of live cells was performed by incubation of cells for 5 minutes with 2 micro-g/ml of Propidium Iodide (PI) and subsequent sorting into PI⁺ and PI⁻ populations with a FACSvantage. PI was also used to exclude dead cells in experiments with cellular stress induced by starvation or gamma-irradiation.

Abbreviations list: ATM, Ataxia Telangiectasia Mutated; BRCT, BRCA C-terminal repeat; NLVS, 4-hydroxy-5-iodo-3-nitrophenylacetyl-Leu-Leu-Leu-vinyl sulphone; PI, Propidium Iodide; PIKKs, Phosphoinositide-3-OH-kinase-related kinases; TPPII, Tripeptidyl-peptidase II; FA=3-(2-furyl)acryloyl.

Standard abbreviations are used for chemicals and amino acids herein.

| Abbreviation | | Alternative abbreviation |
|---|---|---|
| A | Alanine | Ala |
| R | Arginine | Arg |
| N | Asparagine | Asn |
| D | Aspartic acid | Asp |
| C | Cysteine | Cys |
| E | Glutamic Acid | Glu |
| Q | Glutamine | Gln |
| G | Glycine | Gly |
| H | Histidine | His |
| I | Isoleucine | Ile |
| L | Leucine | Leu |
| K | Lysine | Lys |
| M | Methionine | Met |
| F | Phenylalanine | Phe |
| P | Proline | Pro |
| S | Serine | Ser |
| T | Threonine | Thr |
| W | Tryptophan | Trp |
| Y | Tyrosine | Tyr |
| V | Valine | Val |

The invention also makes use of several unnatural alpha-amino acids.

| Abbreviation | | SIDE CHAIN |
|---|---|---|
| Abu | 2-aminobutyric acid | CH₂CH₃ |
| Nva | norvaline | CH₂CH₂CH₃ |
| Nle | norleucine | CH₂CH₂CH₂CH₃ |
| | tert-butyl alanine | CH₂C(CH₃)₃ |
| | alpha-methyl leucine | (CH₃)(CH₂C(CH₃)CH₃) |
| | 4,5-dehydro-leucine | CH₂C(═CH₂)CH₃ |
| | allo-isoleucine | CH(CH₃)CH₂CH₃ |
| | alpha-methyl valine | (CH₃)CH(CH₃)(CH₃) |
| | tert-butyl glycine | C(CH₃)₃ |
| | 2-allylglycine | CH₂CH═CH₂ |
| Orn | Ornithine | CH₂CH₂CH₂NH₂ |
| Dab | alpha,gamma-diaminobutyric | CH₂CH₂NH₂ |
| | acid 4,5-dehydro-lysine | CH₂CH═CHCH₂NH₂ |

### Example 1 and FIG. 1

### TPPII Mediates Stress-Induced Inhibition of Proteasomal Substrate Degradation

To test the rate of proteasomal degradation during cellular stress we used EL-4.Ub-R-GFP and HeLa.UbGV76-GFP cells, stably transfected with Green Fluorescent Protein (GFP)-reporter substrates (Dantuma, N. P, Lindsten, K., Glas, R., Jellne, M., and Masucci, M. G. 2000. Short-lived green fluorescent proteins for quantifying ubiquitin/proteasome-dependent proteolysis in living cells. Nat. Biotechnol. 18:538-43). These GFP-based substrates are N-terminally modified to become rapidly ubiquitinated and degraded by the proteasome. By flow cytometry analysis of live cells, we observed strong increase of steady state levels of GFP-fluorescence in both EL-4.Ub-R-GFP and HeLa.UbGV76-GFP treated with the proteasomal inhibitor NLVS (FIG. 1). In addition, we found that exposure of EL-4.Ub-R-GFP and HeLa.UbGV76-GFP cells to starvation as well as gamma-irradiation led to an accumulation of GFP-fluorescence, reaching levels observed during treatment with low concentrations of NLVS (FIG. 1). Our data thus suggest reduced activity of the ubiquitin-proteasome pathway during cellular stress.

### Example 2 and FIG. 2

The large cytosolic peptidase, tripeptidyl-peptidase II (TPPII) is believed to be important for the transduction of signals from activated members of the family of PI3K-like kinases (PIKKs). Since PIKKs are important for responses to both starvation and gamma-irradiation we tested whether TPPII was important for inhibition of the ubiquitin-proteasome pathway. We therefore made EL-4.UbGV76-GFP cells co-transfected with pSUPER-TPPIIⁱ, a vector we previously used to obtain siRNA-mediated suppression of TPPII expression. By exposure of EL-4.UbGV76-GFP and EL-4.UbGV76-GFP/TPPIIⁱ cells (co-transfected with the TPPIIⁱ siRNA-encoding plasmid) to gamma-irradiation we found that the induction of GFP-fluorescence was at least in part dependent on the expression of TPPII (FIG. 2a). Further, EL-4.TPPIIⁱ cells had a significantly increased ability to proliferate in the presence of higher concentrations of proteasomal inhibitor compared to EL-4.wt cells (expressing empty pSUPER vector, FIG. 2b). This correlated with an increased ability to degrade the fluorescent reporter substrate UbGV76-GFP, in EL-4.UbGV76-GFP cells co-transfected with the TPPIIⁱ siRNA plasmid (FIG. 2c), further suggesting that TPPII inhibits the degradation of proteasomal substrates.

To further substantiate that TPPII reduces proteasomal substrate degradation we studied degradation of a substrate that often resists degradation by the proteasome. We used Ub-R-GFP-Q112, a reporter substrate similar to those used previously, but containing a C-terminal poly-Glutamine repeat (Verhoef, L. G., Lindsten, K., Masucci, M. G. and Dantuma, N. P. 2002. Aggregate formation inhibits proteasomal degradation of polyglutamine proteins. Hum Mol Genet. 11:2689-700). Such poly-Glutamine sequences inhibit proteasomal protein degradation, accumulate in intracellular inclusions and cause neurodegenerative disease (Zoghbi, H. Y. and Orr, H. T. 2000. Glutamine repeats and neurodegeneration. Annu Rev Neurosci. 23:217-47) (Bence, N. F., Sampat, R. M., and Kopito, R. R. 2001. Impairment of the ubiquitin-proteasome system by protein aggregation. Science 292:1552-5) (Venkatraman, P., Wetzel, R., Tanaka, M., Nukina, N., and Goldberg, A. L. 2004. Eukaryotic proteasomes cannot digest polyglutamine sequences and release them during degradation of polyglutamine-containing proteins. Mol Cell. 14:95-104). We found that stably expressing EL-4 cells failed to efficiently degrade Ub-R-GFP-Q112, and treatment with NLVS further increased accumulation of substrate not degraded in EL-4 cells (FIG. 2d). However, co-transfection with the pSUPER-TPPIIⁱ siRNA plasmid allowed complete removal of accumulated Ub-R-GFP-Q112 substrate, which was dependent on the catalytic activity of the 20S proteasome (FIG. 2d). Furthermore, we used two different catalytic inhibitors of TPPII, Butabindide and Z-Gly-Leu-Ala-OH (Z-GLA-OH), to examine whether they affected stability of the R-GFP-Q112 substrate in EL-4 cells. Z-GLA-OH is an inhibitor designed to target its bacterial homologue Subtilisin, an enzyme that shares catalytic mechanism with TPPII (Tomkinson, B. 1999. Tripeptidyl peptidases: enzymes that count. Trends Biochem Sci. 24:355-9) (Bryan, P. N. 2000. Protein engineering of subtilisin. Biochim Biophys Acta. 1543:203-222). When EL-4.Ub-R-GFP-Q112 cells were treated with 100 micro-M Butabindide or 25-microM Z-GLA-OH we observe a gradual decline in EL-4.Ub-R-GFP-Q112 substrate, suggesting that the activity of TPPII suppresses their degradation (FIG. 2e). In these in vitro experiments with peptidase inhibitors we used serum-free AIM-V medium to avoid serum-induced destabilization of our inhibitors (Reits, E., et. al. 2004. A major role for TPPII in trimming proteasomal degradation products for MHC class I antigen presentation. Immunity 20:495-506). From these observations we conclude that TPPII mediated a stress-induced inhibition of the UPP.

### Example 3 and FIG. 3

Starvation-induced nuclear localization of proteasomal complexes requires TPPII expression and activity. To further characterize the mechanism by which TPPII controlled proteasomal substrate degradation we tested if TPPII regulated sub-cellular localization of 19S regulatory proteasomal complexes. We made immuno-histochemical staining using an antibody directed against the Rpt6 ATPase sub-unit, a component of the 19S base complex. In untreated EL-4.wt and EL-4.TPPIIⁱ cells, a homogenous 19S proteasome-staining was clear in the cytosol and nuclei (FIGS. 3a, b, top panels). Further, starving EL-4.wt cells acquired a reduced cellular volume, and strong staining of Rpt6 was detectable in the nucleus, as evident by comparison with Hoechst 33258 controls with characteristically stained nuclei (FIG. 3a, lower panels). In contrast, we find that EL-4 cells with inhibited TPPII expression, EL-4.TPPIIⁱ, failed to localize 19S proteasomes into their nuclei during starvation (FIG. 3b, lower panels). Reduced cell size coincides with cellular sensing of stress, and similar data was obtained during exposure to gamma-irradiation. Release from starvation allowed proliferation of most EL-4.wt as well as EL-4.TPPIIⁱ cells, showing that these cells were not apoptotic (FIG. 3c).

We further purified cytosols of control and stressed EL-4.wt and EL-4.TPPIIⁱ cells, and analyzed the high molecular weight fraction of cytosols biochemically. We found strong expression of proteasomal alpha-3 sub-units in cytosols of both EL-4.wt and EL-4.TPPIIⁱ cells, as detected by western blotting (FIG. 3d). However, after starvation virtually no proteasomes were biochemically detected in cytosols from EL-4.wt whereas similar detection of cytosolic alpha-3 sub-units was detected in EL-4.TPPIIⁱ cells.

### Example 4 and FIG. 4

A TPPII-dependent shift in proteasomal distribution was also found after starvation of HeLa cells (human cervical carcinoma, FIG. 4a). The involvement of TPPII in this process was supported by the use of the TPPII-specific inhibitor butabindide that inhibited nuclear localization of proteasomes in EL-4, HeLa cells (FIGS. 4a, b).

### Example 5 and FIG. 5

### Stress-Induced Kinases Control Proteasomal Distribution and TPPII Expression

During responses to DNA damage the expression of TPPII is controlled by members of the PIKK family. In order to test whether PIKK signaling was required also for nuclear localization of proteasomes in response to starvation, we treated our starved EL-4 cells with 1 micro-M wortmannin, an inhibitor of PIKK family kinases. We found that the stress-induced translocation of proteasomes in EL-4 cells was inhibited by 1 micro-M wortmannin, since this treatment redirected proteasomal complexes into the cytosol (FIGS. 5a, b). We further found that also stress-induced TPPII-expression required PIKK-kinase activity, since 1 micro-M wortmannin incubation induced degradation of all detectable TPPII protein after 6-9 hours, whereas expression of proteasomal alpha-3 sub-units remained constant during the same period (FIG. 5c). However, blocking the proteasome with NLVS prevented rapid TPPII down regulation during wortmannin treatment, suggesting proteasomal degradation of TPPII when stress signaling is inhibited (FIG. 5c). These data suggest that TPPII is a PIKK-induced mediator of stress signals that is required for nuclear localization of proteasomes during cellular starvation.

Our data indicated that signaling by PIKKs during cellular stress inhibits the proteasomal substrate degradation. Inhibition of TPPII allows degradation of a poly-Glutamine substrate in stably transfected cells in vitro. Therefore we propose the treatment, possibly periodically of patients with neurodegenerative diseases to clear the load of pathogenic, disease-provoking, proteins. Further, our experiments reveal in particular the tripeptide TPPII inhibitors as compounds to distribute in this situation.

### Example 6 and FIG. 6

### Requirement for TPP II in Starvation-Induced p53 Accumulation and Growth Arrest

We tested whether TPP II was important in responses to starvation since this type of stress is controlled by PIKKs, and is highly relevant for disease pathogenesis, e.g. in ischemic disease. We tested the expression of TPP II in proliferating cultures of EL-4 cells, and we observed that cell cultures reaching high densities after 4-5 days of proliferation gradually acquired high TPP II expression, while reaching the peak of cell density (FIGS. 6 a and b). Addition of fresh medium in these cell cultures led to rapid down regulation of TPP II expression (indicated by arrow, FIG. 6 a). These data further support that the expression of TPPII is up-regulated by starvation.

We investigated the functional role of TPPII in responses to starvation. We observed down regulation of DNA synthesis in cultures of EL-4.wt control cells approaching maximum cell density, as detected by ³H-Tritium incorporation, and this was not observed in EL-4.TPP IIⁱ cells (FIG. 6 c). In line with these data we find reduced accumulation of p53 in starved EL-4.TPP IIⁱ cells, compared to EL-4.wt cells (FIG. 6 d). To further test for the presence of growth arrest in starving EL-4.wt and EL-4.TPP IIⁱ cells, we made flow cytometric sorting of live cells (i.e. PI^{neg} cells) that survived starvation for 2-5 days, and measured their proliferation in fresh medium. Approximately 50% of these cells were PI^{neg} after 2 days, whereas only 3-5% remained PI^{neg} after 5 days of starvation. We found that cultures of starved Pines EL-4.wtcells had a substantial delay before proliferation resumed, and this was especially apparent among the minor fraction of EL-4.wt cells that survived starvation 5 days (FIG. 6 e). In contrast EL-4.TPP IIⁱ cells that survived 5 days of starvation resumed rapid proliferation almost immediately. Thereby, responses to several types of stress controlled by PIKKs require TPP II. The strong expression of TPPII observed in starved cells clearly contributed to cell cycle arrest and p53 accumulation.

Animal mouse models of stroke show a strongly reduced neuronal death in p53−/− animals (Yonekura I, Takai K, Asai A, Kawahara N, Kirino T. p53 potentiates hippocampal neuronal death caused by global ischemia. J Cereb Blood Flow Metab. 2006, 26:1332-40). Our data show that TPPII inhibition reduces p53 accumulation in response to several forms of stress, e.g. starvation. Therefore we propose TPPII as a target for the treatment of ischemic disease. For example the tripeptide TPPII inhibitors may be administered, for example injected into acutely systemically ill patients, to reduce p53 accumulation in the ischemic tissues, and thereby prolong tissue survival. Provided sufficient time, collateral pathways of blood flow are created, and the inhibition of p53 accumulation may therefore reduce the infarction size, e.g. during stroke.

### Example 7 and FIG. 7

Importantly we have shown that p53 accumulation depends on TPPII activity (FIG. 7a). Further, we find that Z-GLA-OH treatment causes a strong reduction in TPPII expression (FIG. 7b), providing a rationale for how blocking of the active site in most cases gives results similar to that observed when using siRNA to block the expression of the molecule.

### Example 8

### In Vitro Testing of Di- and Tri-Peptides and Derivatives

Table 1 contains in vitro data, in fluorometric units which are arbitrary but relative, for the inhibition of cleavage of AAF-AMC (H-Ala-Ala-7-amido-4-methylcoumarin) by compounds at several concentrations. Some beneficial effect is seen for most of the compounds tested.

TPP II protein was enriched, and then a TPP II-preferred fluorogenic substrate AAF-AMC was used. 100×10⁶ cells were sedimented and lysed by vortexing in glass beads and homogenisation buffer (50 mM Tris Base pH 7.5, 250 mM Sucrose, 5 mM MgCl₂, 1 mM DTT). Cellular lysates were subjected to differential centrifugation; first the cellular homogenate was centrifuged at 14,000 rpm for 15 min, and then the supernatant was transferred to ultra-centrifugation tubes. Next the sample was ultra-centrifugated at 100,000×g for 1 hour, and the supernatant (denoted as cytosol in most biochemical literature) was subjected to 100,000×g centrifugation for 5 hours, which sedimented high molecular weight cytosolic proteins/protein complexes. The resulting pellet dissolved in 50 mM Tris Base pH 7.5, 30% Glycerol, 5 mM MgCl₂, and 1 mM DTT, and 1 ug of high molecular weight protein was used as enzyme in peptidase assays.

To test the activity of TPP II we used the substrate and AAF-AMC (Sigma, St. Louis, Mo.), at 100 uM concentration in 100 ul of test buffer composed of 50 mM Tri Base pH 7.5, 5 mM MgCl₂ and 1 mM DTT. To stop reactions we used dilution with 900 ul 1% SDS solution. Cleavage activity was measured by emission at 460 nm in a LS50B Luminescence Spectrometer (Perkin Elmer, Boston, Mass.).

FA=3-(2-furyl)acryloyl; PBS=phosphate-buffered saline. The text (Z, FA, H, etc.) at the start of each compound name is the substituent at the N-terminus; H indicates that the N-terminus is free NH₂. The text (OH, NBu, etc.) at the end of each compound name is the substituent at the C-terminus: OH indicates that the C-terminus is free CO₂H.

TABLE 1

| | 100 | 10 | 1 | | | | |
|---|---|---|---|---|---|---|---|
| Compound | uM | uM | uM | 100 nM | 10 nM | 1 nM | 0 |
| Z-GL-OH | 23.14 | 23.60 | 24.18 | 34.6 | 34.07 | 44.53 | 49.55 |
| (comparative) | 24.99 | 24.72 | 24.4 | 33.02 | 33.85 | 44.21 | 49.82 |
| | 23.69 | 24.59 | 24.29 | 34.6 | 34.38 | 43.62 | 49.51 |
| mean | 23.94 | 24.30 | 24.29 | 34.07 | 34.1 | 44.12 | 49.63 |
| Z-GLG-OH | 14.44 | 17.49 | 23.79 | 31.49 | 34.4 | 43.42 | 48.58 |
| | 15.02 | 17.58 | 24.85 | 28.64 | 34.16 | 44.02 | 49.03 |
| | 15.8 | 17.44 | 24.63 | 26.13 | 34.27 | 43.73 | 49.2 |
| mean | 15.09 | 17.50 | 24.42 | 28.75 | 34.28 | 43.72 | 48.94 |
| Z-GGA-OH | 15.5 | 16.65 | 21.37 | 24.27 | 36.01 | 43.42 | 51.19 |
| | 15.27 | 17.27 | 22.14 | 31.54 | 36.59 | 43.87 | 48.44 |
| | 15.78 | 17.18 | 22.62 | 31.61 | 36.73 | 44.14 | 48.48 |
| mean | 15.52 | 17.03 | 22.04 | 29.14 | 36.44 | 43.81 | 49.37 |
| FA-GLA-OH | 6.34 | 14.35 | 19.99 | 23.33 | 31.19 | 43.18 | 49.96 |
| | 4.05 | 8.14 | 16.21 | 23.87 | 33.88 | 43.49 | 48.4 |
| | 4.69 | 9.44 | 14.78 | 24.09 | 33.9 | 43.68 | 49.43 |
| mean | 5.03 | 10.64 | 16.99 | 23.76 | 32.99 | 43.45 | 49.26 |
| H-APA-OH | 13.55 | 14.35 | 23.94 | 24.26 | 28.85 | 44.05 | 48.84 |
| | 8.46 | 14.64 | 24.49 | 24.48 | 29.39 | 41.76 | 49.32 |
| | 7.65 | 14.91 | 25.04 | 28.44 | 29.44 | 43.84 | 49.16 |
| mean | 9.89 | 14.63 | 24.49 | 25.73 | 29.23 | 43.22 | 49.11 |
| H-GLA-OH | 8.37 | 12.4 | 15.53 | 17.58 | 22.67 | 36.63 | 48.16 |
| | 7.42 | 12.53 | 19.03 | 17.94 | 23.33 | 38.42 | 49.91 |
| | 7.12 | 14.66 | 18.34 | 17.53 | 22.93 | 39.4 | 48.18 |
| mean | 7.64 | 13.20 | 17.63 | 17.68 | 22.98 | 38.15 | 48.75 |
| Bn-GLA-OH | 12.92 | 17.74 | 21.14 | 23.01 | 33.30 | 43.67 | 48.53 |
| | 11.17 | 14.86 | 21.54 | 22.71 | 33.45 | 42.91 | 47.02 |
| | 9.65 | 13.38 | 22.01 | 22.90 | 33.40 | 41.17 | 49.55 |
| mean | 11.25 | 15.33 | 21.56 | 22.87 | 33.38 | 42.58 | 48.37 |
| Z-GKA-OH | 8.17 | 12.48 | 14.49 | 21.62 | 23.57 | 42.13 | 49.82 |
| | 9.44 | 14.52 | 16.43 | 21.98 | 23.95 | 42.02 | 49 |
| | 9.44 | 14.82 | 15.03 | 21.52 | 24.36 | 42.51 | 47.7 |
| mean | 9.02 | 13.94 | 15.32 | 21.71 | 23.96 | 42.22 | 48.84 |
| Z-GLA-Nbu | 11.16 | 13.06 | 23.89 | 32.24 | 34.06 | 38.14 | 47.34 |
| | 13.86 | 14.73 | 23.71 | 32.41 | 33.89 | 38.31 | 47 |
| | 14.05 | 14.34 | 24.13 | 32.63 | 34.85 | 36.63 | 48 |
| mean | 13.02 | 14.04 | 23.91 | 32.43 | 34.27 | 37.69 | 47.45 |
| Z-GLA-OH | 1.14 | 6.47 | 11.43 | 14.43 | 21.74 | 32.54 | 49 |
| | 1.44 | 7.66 | 11.9 | 14.26 | 21.93 | 32.61 | 49.4 |
| | 1.55 | 7.49 | 11.46 | 14.37 | 24.44 | 33.41 | 49.5 |
| mean | 1.38 | 7.21 | 11.60 | 14.35 | 22.70 | 32.85 | 49.30 |

Other compounds also performed well in the above in vitro test. including GPG-NH₂ and Z-GPG-NH₂.

1. A method of treating a neurodegenerative disease or an ischemic condition comprising administering to a patient in need thereof a therapeutically effective amount of a TPP II inhibitor compound.

2. A method as claimed in claim 1, wherein said compound is selected from formula (i) or is a pharmaceutically acceptable salt thereof:
R^{N1}R^{N2}N-A¹-A²-A³-CO—R^{C1} (i)
wherein A¹, A² and A³ are amino acid residues having the following definitions according to the standard one-letter amino acid abbreviations or names:
A¹ is G, A, V, L, I, P, 2-aminobutyric acid, norvaline or tert-butyl glycine,
A² is G, A, V, L, I, P, F, W, C, S, K, R, 2-aminobutyric acid, norvaline, norleucine, tert-butyl alanine, alpha-methyl leucine, 4,5-dehydro-leucine, allo-isoleucine, alpha-methyl valine, tert-butyl glycine, 2-allylglycine, ornithine or alpha, gamma-diaminobutyric acid,
A³ is G, A, V, L, I, P, F, W, D, E, Y, 2-aminobutyric acid, norvaline or tert-butyl glycine,
R^{N1} and R^{N2} are each attached to the N terminus of the peptide, are the same or different, and are each independently
R^{N3},
(linker1)-R^{N3},
CO-(linker1)-R^{N3},
CO—O-(linker1)-R^{N3},
CO—N-((linker1)-R^{N3})R^{N4} or
SO₂-(linker1)-R^{N3},
(linker1) may be absent, i.e. a single bond, or CH₂, CH₂CH₂, CH₂CH₂CH₂, CH₂CH₂CH₂CH₂ or CH═CH,
R^{N3} and R^{N4} are the same or different and are hydrogen or any of the following optionally substituted groups:
saturated or unsaturated, branched or unbranched C₁₋₆ alkyl;
saturated or unsaturated, branched or unbranched C₃₋₁₂ cycloalkyl;
benzyl;
phenyl;
naphthyl;
mono- or bicyclic C₁₋₁₀ heteroaryl; or
non-aromatic C₁₋₁₀ heterocyclyl;
wherein there may be zero, one or two (same or different) optional substituents on R^{N3} and/or R^{N4} which may be:
hydroxy-;
thio-:
amino-;
carboxylic acid;
saturated or unsaturated, branched or unbranched C₁₋₆ alkyloxy;
saturated or unsaturated, branched or unbranched C₃₋₁₂ cycloalkyl;
N—, O—, or S— acetyl;
carboxylic acid saturated or unsaturated, branched or unbranched C₁₋₆ alkyl ester;
carboxylic acid saturated or unsaturated, branched or unbranched C₃₋₁₂ cycloalkyl ester
phenyl;
mono- or bicyclic C₁₋₁₀ heteroaryl;
non-aromatic C₁₋₁₀ heterocyclyl; or
halogen; and
R^{C1} is attached to the C terminus of the tripeptide, and is:
O—R^{C2},
O-(linker2)-R^{C2},
N((linker2)R^{C2})R^{C3}, or
N(linker2)R^{C2}—NR^{C3}R^{C4}
(linker2) may be absent, i.e. a single bond, or C₁₋₆ alkyl or C₂₋₄ alkenyl;
wherein R^{C2}, R^{C3} and R^{C4} are the same or different, and are hydrogen or any of the following optionally substituted groups:
saturated or unsaturated, branched or unbranched C₁₋₆ alkyl;
saturated or unsaturated, branched or unbranched C₃₋₁₂ cycloalkyl;
benzyl;
phenyl;
naphthyl;
mono- or bicyclic C₁₋₁₀ heteroaryl; or
non-aromatic C₁₋₁₀ heterocyclyl;
wherein there may be zero, one or two same or different optional substituents on each of R^{C2} and/or R^{C3} and/or R^{C4} which may be one or more of:
hydroxy-;
thio-:
amino-;
carboxylic acid;
saturated or unsaturated, branched or unbranched C₁₋₆ alkyloxy;
saturated or unsaturated, branched or unbranched C₃₋₁₂ cycloalkyl;
N—, O—, or S— acetyl;
carboxylic acid saturated or unsaturated, branched or unbranched C₁₋₆ alkyl ester;
carboxylic acid saturated or unsaturated, branched or unbranched C₃₋₁₂ cycloalkyl ester
phenyl;
halogen;
mono- or bicyclic C₁₋₁₀ heteroaryl; or
non-aromatic C₁₋₁₀ heterocyclyl.

3. A method as claimed in claim 2 wherein said compound of formula (i) is such that:
R^{N1} is hydrogen,
R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.

4. A method as claimed in claim 3, wherein said compound of formula (i) is such that:
A¹ is G, A or 2-aminobutyric acid,
A² is L, I, norleucine, V, norvaline, tert-butyl alanine, 4,5-dehydro-leucine, allo-isoleucine, 2-allylglycine, P, 2-aminobutyric acid, alpha-methyl leucine, alpha-methyl valine or tert-butyl glycine,
A³ is G, A, V, P, 2-aminobutyric acid or norvaline,
R^{N1} is H,
R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.

5. A method as claimed in claim 4, wherein said compound of formula (i) is such that:
A¹ is G, A or 2-aminobutyric acid,
A² is L, I, norleucine, V, norvaline, tert-butyl alanine, 4,5-dehydro-leucine, allo-isoleucine or 2-allylglycine,
A³ is G, A, V, P, 2-aminobutyric acid or norvaline,
R^{N1} is H,
R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.

6. A method as claimed in claim 5 wherein said compound of formula (i) is such that:
A¹ is G or A,
A² is L, I, or norleucine,
A³ is G or A,
R^{N1} is hydrogen,
R^{N2} is hydrogen, C(═O)—O-saturated or unsaturated, branched or unbranched, C₁₋₄ alkyl, optionally substituted with phenyl or 2-furyl, or C(═O)— saturated or unsaturated, branched or unbranched, C₁₋₄ alkyl, optionally substituted with phenyl or 2-furyl, and
R^{C1} is OH, O—C₁₋₆ alkyl, O—C₁₋₆ alkyl-phenyl, NH—C₁₋₆ alkyl, or NH—C₁₋₆ alkyl-phenyl.

7. A method as claimed in claim 2 wherein
R^{N1} is hydrogen,
R^{N2} is hydrogen, C(═O)—OCH₂Ph or C(═O)—CH═CH-(2-furyl), and
R^{C1} is OH, O—C₁₋₆ alkyl, or NH—C₁₋₆ alkyl.

8. A method as claimed in claim 7 wherein said compound of formula (i) is
Z-GLA-OH, Bn-GLA-OH, FA-GLA-OH or H-GLA-OH.

9. A method as claimed in claim 8 wherein said compound of formula (i) is
Z-GLA-OH

10. A method as claimed in claim 2 wherein A¹ is G, A or 2-aminobutyric acid.

11. A method as claimed in claim 10 wherein A¹ is G or A.

12. A method as claimed in claim 2 wherein A² is L, I, norleucine, V, norvaline, tert-butyl alanine, 4,5-dehydro-leucine, allo-isoleucine, 2-allylglycine, P, K, 2-aminobutyric acid, alpha-methyl leucine, alpha-methyl valine or tert-butyl glycine.

13. A method as claimed in claim 12 wherein A² is L, I, norleucine, V, norvaline, tert-butyl alanine, 4,5-dehydro-leucine, allo-isoleucine, 2-allylglycine, P or K.

14. A method as claimed in claim 13 wherein A² is L, I, norleucine, P or K.

15. A method as claimed in claim 14 wherein A² is L or P.

16. A method as claimed in claim 15 wherein A² is P.

17. A method as claimed in claim 2 wherein A³ is G, A, V, P, 2-aminobutyric acid or norvaline.

18. A method as claimed in claim 17 wherein A³ is G or A.

19. A method as claimed in claim 2 wherein R^{N1} is hydrogen.

20. A method as claimed in claim 2 wherein R^{N2} is
R^{N3},
(linker1)-R^{N3},
CO-(linker1)-R^{N3}, or
CO—O-(linker1)-R^{N3},
wherein
(linker1) is a single bond, or CH₂, CH₂CH₂, CH₂CH₂CH₂, CH₂CH₂CH₂CH₂ or CH═CH, and
R^{N3} is hydrogen or any of the following unsubstituted groups:
saturated or unsaturated, branched or unbranched C₁₋₄ alkyl;
benzyl;
phenyl; or
monocyclic heteroaryl.

21. A method as claimed in claim 20 wherein R^{N2} is hydrogen, benzyloxycarbonyl, benzyl, benzoyl, tert-butyloxycarbonyl, 9-fluorenylmeth-oxycarbonyl or FA.

22. A method as claimed in claim 21 wherein R^{N2} is hydrogen, benzyloxycarbonyl or FA.

23. A method as claimed in claim 2 wherein R^{C1} is:
O—R^{C2},
O-(linker2)-R^{C2}, or
NH-(linker2)R^{C2}
wherein
(linker2) is a single bond, C₁₋₆ alkyl or C₂₋₄ alkenyl, preferably a single bond or CH₂, CH₂CH₂, CH₂CH₂CH₂, CH₂CH₂CH₂CH₂ or CH═CH, and
R^{C2} is hydrogen or any of the following unsubstituted groups:
saturated or unsaturated, branched or unbranched C₁₋₅ alkyl;
benzyl;
phenyl; or
monocyclic C₁₋₁₀ heteroaryl.

24. A method as claimed in claim 23 wherein R^{C1} is OH, O—C₁₋₆ alkyl, O—C₁₋₆ alkyl-phenyl, NH₂, NH—C₁₋₆ alkyl, or NH—C₁₋₆ alkyl-phenyl.

25. A method as claimed in claim 24 wherein R^{C1} is OH, O—C₁₋₆ alkyl, NH₂, or NH—C₁₋₆ alkyl.

26. A method as claimed in claim 25 wherein R^{C1} is OH or NH₂.

27. A method as claimed in claim 26 wherein R^{C1} is NH₂.

28. A method as claimed in claim 2 wherein said compound is GPG-NH₂, Z-GPG-NH₂, Bn-GPG-NH₂, FA-GPG-NH₂, GPG-OH, Z-GPG-OH, Bn-GPG-OH, or FA-GPG-OH.

29. A method as claimed in claim 28 wherein said compound is GPG-NH₂.

30. A method as claimed in claim 2 wherein said compound is ALG-NH₂, Z-ALG-NH₂, Bn-ALG-NH₂, FA-ALG-NH₂, ALG-OH, Z-ALG-OH, Bn-ALG-OH, or FA-ALG-OH.

31. A method as claimed in claim 30 wherein said compound is ALG-NH₂.

32. A method as claimed in claim 2 wherein A³ is not F, W, D, E or Y.

33. A method as claimed in claim 2 wherein A³ is not P.

34. A method as claimed in claim 2 wherein A³ is not E.

35-39. (canceled)

40. A method of treatment of a neurodegenerative disease comprising administering to a patient in need thereof a therapeutically effective amount of a compound defined in claim 1.

41. A method of treatment of a neurodegenerative disease selected from Alzheimer's, Parkinson's or Huntingdon's disease comprising administering to a patient in need thereof a therapeutically effective amount of a compound defined in claim 1.

42. A method treatment of an ischemic condition comprising administering to a patient in need thereof a therapeutically effective amount of a compound defined in claim 1.

43. A method of treatment of an ischemic condition selected from stroke and cardiac infarction comprising administering to a patient in need thereof a therapeutically effective amount of a compound defined in claim 1.

44-48. (canceled)

49. A method for identifying a compound suitable for the treatment of a neurodegenerative disease comprising contacting TPP II with a compound to be screened, and identifying whether the compound inhibits the activity of TPP II.

50. A method of identifying a compound suitable for the treatment of a neurodegenerative disease comprising contacting TPP II with a compound to be screened, and identifying whether the compound inhibits the activity of TPP II.

51. A method for identifying a compound suitable for the treatment of a neurodegenerative disease selected from Alzheimer's, Parkinson's or Huntingdon's disease comprising contacting TPP II with a compound to be screened, and identifying whether the compound inhibits the activity of TPP II.

52. A method for identifying a compound suitable for the treatment of an ischemic condition comprising contacting TPP II with a compound to be screened, and identifying whether the compound inhibits the activity of TPP II.

53. A method for identifying a compound suitable for the treatment of an ischemic condition selected from stroke and cardiac infarction comprising contacting TPP II with a compound to be screened, and identifying whether the compound inhibits the activity of TPP II.

54. Pharmaceutical composition comprising a compound with a structure of formulae (i) as defined in claim 2 and a pharmaceutically acceptable diluent or carrier.

55. Pharmaceutical composition comprising a compound with a structure as defined in claim 2 and a pharmaceutically acceptable diluent or carrier wherein said compound is not cinnamoyl-IFP-ethylamide, GPE-OH, GGF-OH, GVF-OH, AAA-OH or IPI-OH.

56. Pharmaceutical composition as claimed in claim 44 with the proviso that A³ is not proline.

57. Pharmaceutical composition as claimed in claim 44 with the proviso that the compound is not GPE-OH.

58. Pharmaceutical composition as claimed in claim 44 with the proviso that R^{C1} is not NH₂.

59. (canceled)
